(19) United States
(12) Patent Application Publication
(10) Pub. No.: US 2026/0182838 A1
(43) Pub. Date: Jul. 2, 2026

(54) LIVING BODY DETECTION APPARATUS

(71) Applicant: SEIKO EPSON CORPORATION, Tokyo (JP)

(72) Inventor: Hiromu TAKAYAMA, Chino-shi (JP)

(73) Assignee: SEIKO EPSON CORPORATION, Tokyo (JP)

(21) Appl. No.: 19/432,211

(22) Filed: Dec. 24, 2025

(30) Foreign Application Priority Data
Dec. 26, 2024 (JP) 2024-230027

(51) Int. Cl. A61B 5/00

(52) U.S. Cl. CPC A61B 5/0059

(57) ABSTRACT

A living body detection apparatus includes a light emitter configured to emit first light; a light receiver configured to receive second light that is the first light reflected from a detection target living body; and a transmissive reflector on which the first light is incident, which is configured to reflect part of the first light toward the detection target living body, on which the second light is incident, and which is configured to transmit part of the second light, and the second light passing through the transmissive reflector is incident on the light receiver.

The present application is based on, and claims priority from JP Application Serial Number 2024-230027, filed December 26, 2024, the disclosure of which is hereby incorporated by reference herein in its entirety.

### BACKGROUND

### 1. Technical Field

The present disclosure relates to a living body detection apparatus that optically detects living body information.

### 2. Related Art

There is a known self-luminous sensor apparatus including a irradiation unit and a light receiver provided on a substrate, the irradiation unit configured to irradiate an object under examination with multiple light beams having different wavelengths in a way that the multiple light beams at least partially overlap with each other, and the light receiver configured to detect light from the object under examination resulting from the multiple irradiation light beams on a wavelength basis (WO 2008/065699). The irradiation unit is configured, for example, with laser diodes that output light beams having multiple wavelengths, and light emission points are arranged side by side on a substrate in a vertical direction corresponding to the thickness of the substrate, the substrate extending in a direction substantially perpendicular to the direction in which the light beams are output.

WO 2008/65699 is an example of the related art.

Since the apparatus described above includes what is called a surface emitting irradiation unit, the light emission points need to be stacked on each other to achieve multi-color emission, so that further size reduction is not achievable.

### SUMMARY

A living body detection apparatus according to an aspect of the present disclosure includes: a light emitter configured to emit first light; a light receiver configured to receive second light that is the first light reflected from a detection target living body; and a transmissive reflector on which the first light is incident, which is configured to reflect part of the first light toward the detection target living body, on which the second light is incident, and which is configured to transmit part of the second light, and the second light passing through the transmissive reflector is incident on the light receiver.

A living body detection apparatus according to another aspect of the present disclosure includes: a light emitter configured to emit first light; a light receiver configured to receive second light that is the first light reflected from a detection target living body; and a transmissive reflector on which the first light is incident, which is configured to transmit part of the first light toward the detection target living body, on which the second light is incident, and which is configured to reflect part of the second light, and the second light reflected from the transmissive reflector is incident on the light receiver.

### BRIEF DESCRIPTION OF THE DRAWINGS

FIG. 1 is a conceptual side cross-sectional view illustrating a living body detection apparatus according to a first embodiment.

FIG. 2 is a conceptual cross-sectional view illustrating the living body detection apparatus taken along a line A-A.

FIG. 3 is a conceptual side cross-sectional view illustrating a variation of the living body detection apparatus shown in FIG. 1.

FIG. 4 is a conceptual side cross-sectional view illustrating a living body information measurement apparatus.

FIG. 5 is a conceptual side cross-sectional view illustrating a living body detection apparatus according to a second embodiment.

FIG. 6 is a conceptual cross-sectional view illustrating the living body detection apparatus taken along a line B-B.

FIG. 7 is a conceptual side cross-sectional view illustrating a living body detection apparatus according to a third embodiment.

FIG. 8 is a conceptual cross-sectional view illustrating the living body detection apparatus taken along a line C-C.

FIG. 9 is a conceptual side cross-sectional view illustrating a living body detection apparatus according to a fourth embodiment.

FIG. 10 is a conceptual cross-sectional view illustrating the living body detection apparatus taken along a line D-D.

FIG. 11 is a conceptual side cross-sectional view illustrating a living body detection apparatus according to a fifth embodiment.

FIG. 12 is a conceptual cross-sectional view illustrating the living body detection apparatus taken along a line E-E.

FIG. 13 is a conceptual cross-sectional view illustrating a variation of the living body detection apparatus shown in FIG. 11.

FIG. 14 is a conceptual side cross-sectional view illustrating a living body detection apparatus according to a sixth embodiment.

FIG. 15 is a conceptual cross-sectional view illustrating the living body detection apparatus taken along a line F-F.

FIG. 16 is a conceptual side cross-sectional view illustrating a variation of the living body detection apparatus shown in FIG. 14.

FIG. 17 is a conceptual cross-sectional view illustrating the living body detection apparatus shown in FIG. 16 taken along a line G-G.

FIG. 18 is a conceptual side cross-sectional view illustrating another variation of the living body detection apparatus shown in FIG. 14.

FIG. 19 is a conceptual side cross-sectional view illustrating still another variation of the living body detection apparatus shown in FIG. 14.

### DESCRIPTION OF EMBODIMENTS

### First embodiment

A living body detection apparatus according to a first embodiment of the present disclosure will be described below with reference to the drawings.

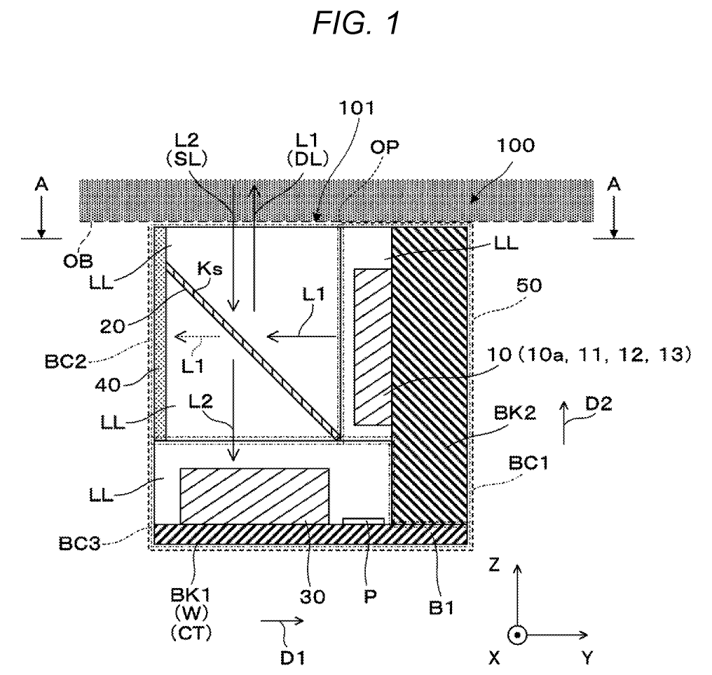

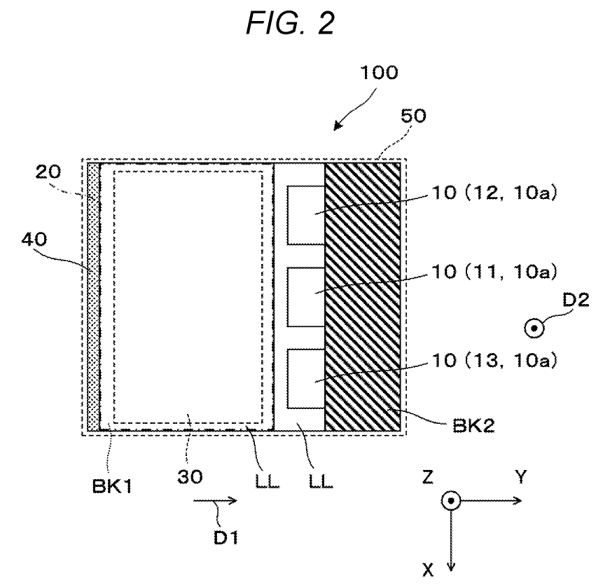

FIG. 1 is a conceptual side cross-sectional view illustrating a living body detection apparatus 100. FIG. 2 is a conceptual cross-sectional view illustrating the living body detection apparatus 100 taken along the line A-A.

The living body detection apparatus 100 shown in FIGS. 1 and 2 is an apparatus that detects living body information on a detection target living body OB by using an optical, non-invasive method. Examples of the detection target living body OB may include a human and an animal. Examples of the living body information may include the pulse and the blood oxygen saturation (blood oxygen concentration). The living body detection apparatus 100 is, for example, a photo-plethysmography (PPG) sensor, and is incorporated, for example, in a vital instrument.

In the following embodiment, the living body detection apparatus 100 will be described as an apparatus that detects living body information on a human. The living body detection apparatus 100 is pressed against the skin of a human and outputs first light L1, which is light having a predetermined wavelength band, toward the skin. The living body detection apparatus 100 receives second light L2, which is the first light L1 reflected from the skin, and detects the pulse, the blood oxygen saturation, or any other parameter based on a temporal change in the amount of the received second light L2. Substances that reflect the first light L1 in the human body are, for example, in-capillary hemoglobin.

The living body detection apparatus 100 has a substantially hexahedral outer shape as a whole, as shown in FIGS. 1 and 2. The living body detection apparatus 100 includes a first substrate BK1, a second substrate BK2, a light emitter 10, a transmissive reflector 20, a light receiver 30, and a light absorbing layer 40. The living body detection apparatus 100 also includes a circuit substrate W, which is a portion incorporated in the first substrate BK1 or the second substrate BK2 and operates the light emitter 10 and the light receiver 30. The living body detection apparatus 100 may or may not be housed in a housing member 50 having an opening OP. The opening OP may be provided with a window that is not shown.

The first substrate BK1 is a plate-shaped member extending in a first direction D1 in a side cross-sectional view parallel to the direction in which the light is projected and received. The second substrate BK2 is a plate-shaped member extending in a second direction D2, which intersects with the first direction D1, in the same side cross-sectional view. In the present embodiment, the first direction D1 is a Y direction and corresponds to the rightward-leftward direction in the plane of view of FIG. 1. The second direction D2 is a Z direction and corresponds to the upward-downward direction in the plane of view of FIG. 1. In the example shown in FIG. 1, the second substrate BK2 extends perpendicularly from an end portion B1 on one side of the first substrate BK1 in the first direction D1. The configuration in which the first substrate BK1 and the second substrate BK2 intersect with each other allows efficient arrangement of the light emitter 10 and the light receiver 30. The first substrate BK1 and the second substrate BK2 are made of a material that has low thermal conductivity and blocks light.

The light emitter 10 is provided at the second substrate BK2. The light receiver 30 is provided at the first substrate BK1. The transmissive reflector 20 is obliquely provided at a position where the first light L1 incident from the light emitter 10 and the second light L2 to be incident on the light receiver 30 intersect with each other.

The living body detection apparatus 100 is provided with a light transmissive member LL in the internal space excluding the first substrate BK1, the second substrate BK2, the light emitter 10, the transmissive reflector 20, the light receiver 30, and the light absorbing layer 40. The transmissive reflector 20 in an inclining posture is surrounded by the light transmissive member LL. The light emitter 10 and the light receiver 30 are embedded and disposed in outer portions of the light transmissive member LL. The first substrate BK1, the second substrate BK2, and the light absorbing layer 40 are disposed so as to cover the outer side of the light transmissive member LL.

The light emitter 10 emits the first light L1. The first light L1 emitted from the light emitter 10 is incident on the transmissive reflector 20. The light emitter 10 includes a surface-emission-type light emitting element 10a. Therefore, even when the light emitter 10 performs multi-color emission, it is unnecessary to stack light emitting elements to form a multi-wavelength light emitter, so that the size of the light emitter 10 can be reduced. The light emitter 10 is disposed at a position shifted from the transmissive reflector 20 in the first direction D1, specifically, in the +Y direction or on the right side of the plane of view of FIG. 1.

The light emitter 10 includes one or more light emitters. In the example shown in FIG. 1, the light emitter 10 includes three light emitters 11 to 13. The light emitters 11 to 13 are arranged in a single direction, specifically, in the X direction. Specifically, the light emitter 10 includes the first light emitter 11, which emits light having a first wavelength band, the second light emitter 12, which emits light having a second wavelength band, and the third light emitter 13, which emits light having a third wavelength band. Multiple pieces of living body information can be detected by controlling the operation of driving the light emitters 11 to 13. The first wavelength band is, for example, a green wavelength band and is used to perform pulse measurement. The green wavelength band ranges, for example, from 500 nm to 570 nm, and is preferably 520 nm. The second wavelength band is, for example, a red wavelength band and is used to perform blood oxygen saturation measurement. The red wavelength band ranges, for example, from 630 nm to 680 nm, and is preferably 660 nm. The third wavelength band is, for example, an infrared wavelength band and is used to perform blood oxygen saturation measurement. The infrared wavelength band ranges, for example, from 850 nm to 1000 nm, and is preferably 905 nm.

Examples of the light emitter 10 may include a light emitting diode (LED), an organic light emitting diode (OLED), a micro-LED, and a vertical cavity surface emitting laser (VCSEL).

The transmissive reflector 20 receives the first light L1 from the light emitter 10, reflects part of the first light L1 toward the detection target living body OB, receives the second light L2 from the detection target living body OB, and transmits part of the second light L2. The second light L2 having passed through the transmissive reflector 20 is incident on the light receiver 30. The transmissive reflector 20 transmits or reflects the first light L1, for example, at a transmittance or reflectance of 50%. That is, the transmissive reflector 20 transmits 50% of the first light L1 and reflects 50% thereof. The transmissive reflector 20 transmits or reflects the second light L2, for example, at a transmittance or reflectance of 50%. That is, the transmissive reflector 20 transmits 50% of the second light L2 and reflects 50% thereof. The reflectance at which the transmissive reflector 20 reflects the first light L1, the transmittance at which the transmissive reflector 20 transmits the second light L2, and other factors of the transmissive reflector 20 can be changed as appropriate.

The transmissive reflector 20 is obliquely disposed with respect to the direction in which the light emitter 10 emits light so as to direct the first light L1 toward the detection target living body OB. The inclination angle of the transmissive reflector 20 is, for example, 45°. The inclination angle of the transmissive reflector 20 can be changed as appropriate. The transmissive reflector 20 is, for example, a membrane-shaped, film-shaped, or plate-shaped member, and is formed on an inclining surface Ks of the light transmissive member LL provided below the transmissive reflector 20. The light transmissive member LL will be described later. The transmissive reflector 20 is made of a material having a transmittance of, for example, about 50%, and is specifically configured with a thin metal film, a cell-cast acrylic plate, a semi-transmissive liquid crystal panel, or the like. The transmissive reflector 20 is produced, for example, by photolithography, electron beam lithography, nanoimprinting, or any other method.

Note that the transmissive reflector 20 may be a triangular-prism-shaped member that has the inclining surface Ks and replaces the light transmissive member LL below the transmissive reflector 20. In this case, the transmissive reflector 20 is made, for example, of a material having a transmittance of about 50%, specifically, polycarbonate, acrylic, polyester, or the like.

The light receiver 30 receives the second light L2, which is the first light L1 reflected from the detection target living body OB. In the present embodiment, the light receiver 30 is disposed at a position shifted from the transmissive reflector 20 in the second direction D2, specifically, in the −Z direction or on the lower side of the plane of view of FIG. 1.

The light receiver 30 handles the light having the wavelength band of the first light L1 emitted from the light emitter 10. Specifically, the light receiver 30 detects the light having the first wavelength band, the light having the second wavelength band, and the light having the third wavelength band.

The light receiver 30 may, for example, be a photodiode.

The light absorbing layer 40 is disposed on the side opposite the light emitter 10 with the transmissive reflector 20 interposed therebetween. The light absorbing layer 40 absorbs the first light L1 having passed through the transmissive reflector 20. The light absorbing layer 40 prevents the first light L1 emitted from the light emitter 10 and passing through the transmissive reflector 20 from being directly incident on the light receiver 30. Noise can thus be suppressed. The light absorbing layer 40 is made, for example, of a black resin material, has low thermal conductivity, and blocks light.

The light transmissive member LL is made of resin, glass, or the like. The light transmissive member LL efficiently transmits the first light L1 and the second light L2.

The circuit substrate W is mounted, for example, on the first substrate BK1. In addition to the light receiver 30, the circuit substrate W includes a drive circuit P, which operates the light emitter 10 and the light receiver 30. The drive circuit P outputs information on an optical response from the detection target living body OB, specifically, the pulse, the blood oxygen saturation, and the like, for example, to a control circuit apparatus 202 of a living body information measurement apparatus 200, which will be described later and in which the living body detection apparatus 100 is incorporated (see FIG. 4). Note that the circuit substrate W may be mounted on the second substrate BK2, may be mounted on both the first substrate BK1 and the second substrate BK2, or may be separate from the first substrate BK1 and the second substrate BK2.

In the living body detection apparatus 100, a member that comes into contact with the detection target living body OB is an insulating member that prevents electricity from conducting from the living body detection apparatus 100 to the detection target living body OB and vice versa. In the present embodiment, the member that comes into contact with the detection target living body OB is the light transmissive member LL exposed via an opening 101 of the living body detection apparatus 100 or the opening OP of the housing member 50.

An example of a method for producing the living body detection apparatus 100 will be described below. The living body detection apparatus 100 can be divided, for example, into three blocks BC1 to BC3. The blocks BC1 to BC3 are first produced, and joined to each other into the assembly of the living body detection apparatus 100. Specifically, the living body detection apparatus 100 includes a first block BC1 including the light emitter 10 and the second substrate BK2, a second block BC2 including the transmissive reflector 20 and the light absorbing layer 40, and a third block BC3 including the light receiver 30 and the first substrate BK1. Note that the members contained in each of the blocks BC1 to BC3 can be changed as appropriate. The shape of each of the blocks BC1 to BC3 and the number of the blocks can also be changed as appropriate.

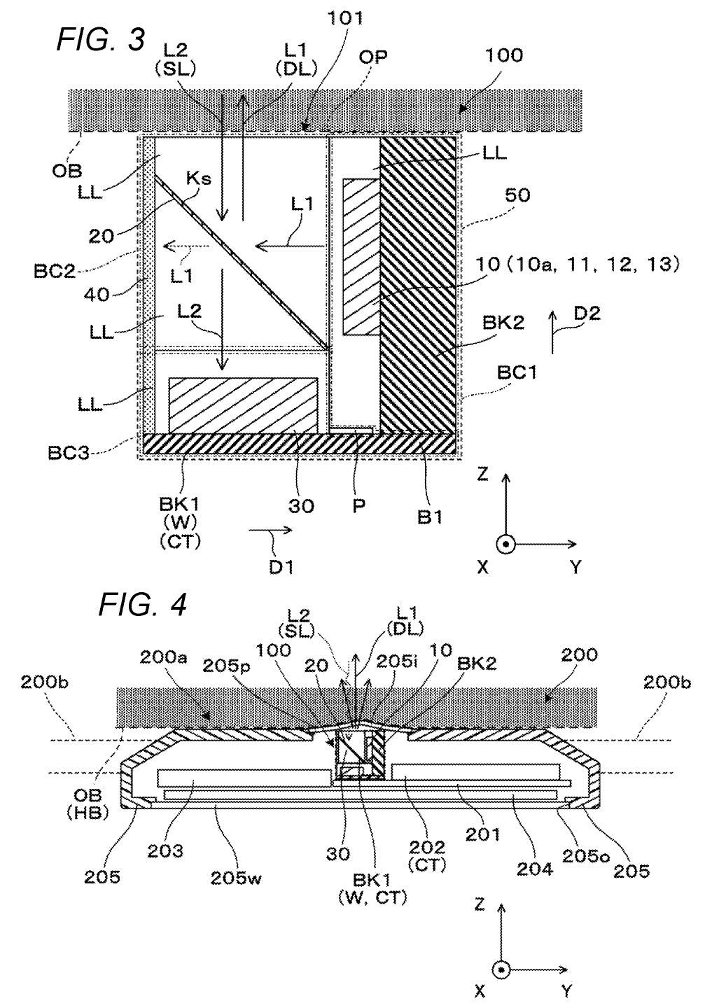

In the first block BC1, the second substrate BK2 may not be exposed but the entirety thereof may be covered with the light transmissive member LL, and the second block BC2 and the third block BC3 may have the same length in the Y direction, as shown in FIG. 3.

The light transmissive member LL contained in each of the blocks BC1 to BC3 is formed, for example, by molding using a mold. The light transmissive members LL contained in the blocks BC1 to BC3 are desirably made of the same material.

The optical path and the like of the living body detection apparatus 100 will be described below. The light emitter 10 emits the first light L1, which is irradiation light DL, toward the detection target living body OB via the opening 101 or the opening OP, as shown in FIG. 1. The first light L1 emitted from the light emitter 10 is reflected from the transmissive reflector 20 by about 50% and is incident on the detection target living body OB. The remaining 50% of the first light L1 passes through the transmissive reflector 20 and is absorbed by the light absorbing layer 40. The first light L1 having entered the detection target living body OB is reflected from the detection target living body OB and becomes the second light L2.

The second light L2 reflected from the detection target living body OB passes through the transmissive reflector 20 by about 50% and is received by the light receiver 30. The light receiver 30 receives the second light L2, which is return light SL having returned from the detection target living body OB via the opening 101 or the opening OP, and outputs a signal corresponding to the intensity of the second light L2.

An example of the operation of the living body detection apparatus 100 will be described below. In the living body detection apparatus 100, when the light emitter 10 includes two or more light emitters, the drive circuit P drives the light emitter 10, specifically, the light emitters 11 to 13, for example, in a time-division manner. The drive circuit P acquires a signal from the light receiver 30 to synchronize the operation of driving the light receiver 30 with the operation of driving the light emitter 10, for example, in a time-division manner. For example, in the pulse measurement, the living body detection apparatus 100 drives only the first light emitter 11, which emits the light having the green first wavelength, and receives the light via the light receiver 30. In the blood oxygen saturation measurement, the living body detection apparatus 100 drives the second light emitter 12, which emits the light having the red second wavelength, and the third light emitter 13, which emits the light having the infrared third wavelength, and receives the light via the light receiver 30. The operation described above prevents the first light L1 from the light emitter 10, which is used for the different application, from becoming noise. In addition, power consumption can be reduced by driving the light emitters 11 to 13, for example, in a time-division manner.

An example of the vital instrument, in which the living body detection apparatus 100 is incorporated, will be described below. Examples of the vital instrument attached to a human body for use may include a smart watch, an active tracker, a smart ring, and a pulse oximeter. In addition, examples of the vital instrument attached to a human contact object for use may include a wireless mouse, a doorknob sensor, and a sensor for a door handle of a vehicle.

FIG. 4 is a conceptual side cross-sectional view illustrating an example of the living body information measurement apparatus 200, in which the living body detection apparatus 100 is incorporated.

The living body information measurement apparatus 200 is a wearable apparatus, has a wristwatch exterior appearance, and can be worn on an arm that is a portion of a human body, that is, a living body HB. The living body information measurement apparatus 200 includes a body apparatus 200a and a pair of wristbands 200b attached to the body apparatus 200a and extending in opposite directions. The body apparatus 200a includes the living body detection apparatus 100, a body substrate 201, the control circuit apparatus 202, a battery 203, a display 204, and an enclosure 205.

The body substrate 201 supports the living body detection apparatus 100 on the side facing the detection target living body OB. The body substrate 201 further supports the control circuit apparatus 202 and the battery 203 on the side opposite the living body detection apparatus 100.

The living body detection apparatus 100 is covered with a plate-shaped window member 205i, which is a light transmissive member and is fitted into an opening 205p, in the enclosure 205. In the example shown in FIG. 4, the window member 205i is slightly curved so as to be convex toward the detection target living body OB.

The control circuit apparatus 202 is an arithmetic processing circuit including a microprocessor, operates the living body detection apparatus 100, and measures living body information based on a value detected by the light receiver 30. The control circuit apparatus 202 and the circuit substrate W are collectively referred to as a controller CT. The control circuit apparatus 202 may be incorporated in the circuit substrate W. The control circuit apparatus 202 or the controller CT acquires living body information such as the pulse and the blood oxygen saturation of the detection target living body OB based, for example, on the second light L2 received by the light receiver 30. In this process, the control circuit apparatus 202 can also correct the value detected by the light receiver 30. The control circuit apparatus 202 can cause the display 204 to display the result of the measurement made by the living body detection apparatus 100. The control circuit apparatus 202 may include a communication circuit and an antenna that allow digital communication with an external instrument.

The battery 203 supplies power to the living body detection apparatus 100 and the control circuit apparatus 202 to operate the living body detection apparatus 100 and the control circuit apparatus 202.

The display 204 is a liquid crystal panel or an organic EL display, performs display operation under the control of the control circuit apparatus 202, and displays various pieces of information such as a result of the measurement. The display 204 is disposed in the enclosure 205 on a side opposite the living body detection apparatus 100 with the body substrate 201 interposed therebetween, and is covered with a plate-shaped window member 205w, which is a light transmissive member and is fitted into an opening 205o.

The living body detection apparatus 100 described above includes the light emitter 10, which emits the first light L1, the light receiver 30, which receives the second light L2, which is the first light L1 reflected from the detection target living body OB, and the transmissive reflector 20, on which the first light L1 is incident, which reflects part of the first light L1 toward the detection target living body OB, on which the second light L2 is incident, and which transmits part of the second light L2, and the second light L2 having passed through the transmissive reflector 20 is incident on the light receiver 30.

According to the living body detection apparatus 100 described above, since the light emitter 10 is so disposed that the light therefrom is reflected from the transmissive reflector 20, and the light receiver 30 is disposed so as to receive the light passing through the transmissive reflector 20, one port (specifically, opening 101) serves as the light entrance and the light exit, so that the area of the surface where the living body detection apparatus 100 comes into contact with the detection target living body OB can be reduced. The size of the living body detection apparatus can thus be reduced.

### Second embodiment

A living body detection apparatus according to a second embodiment of the present disclosure will be described below. Note that the living body detection apparatus according to the second embodiment is a partly changed version of the living body detection apparatus according to the first embodiment, and portions common to those of the living body detection apparatus according to the first embodiment will not be described.

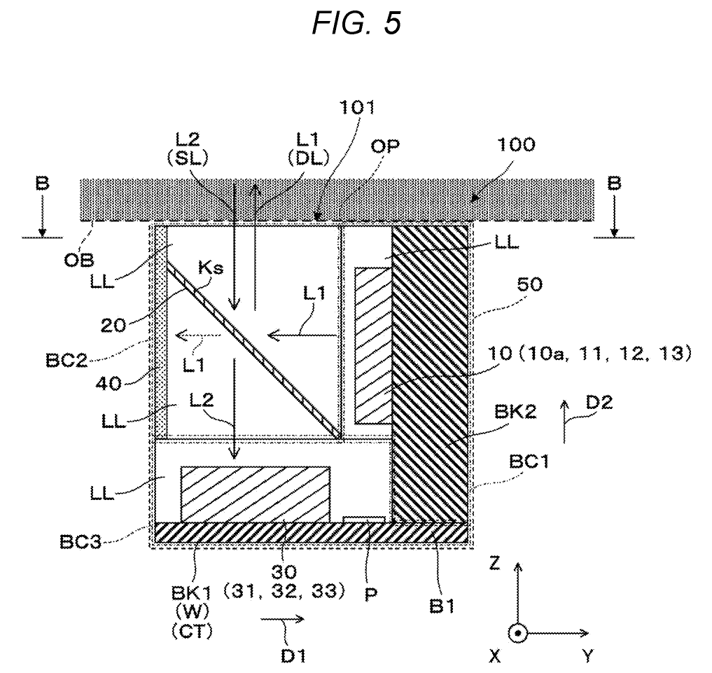

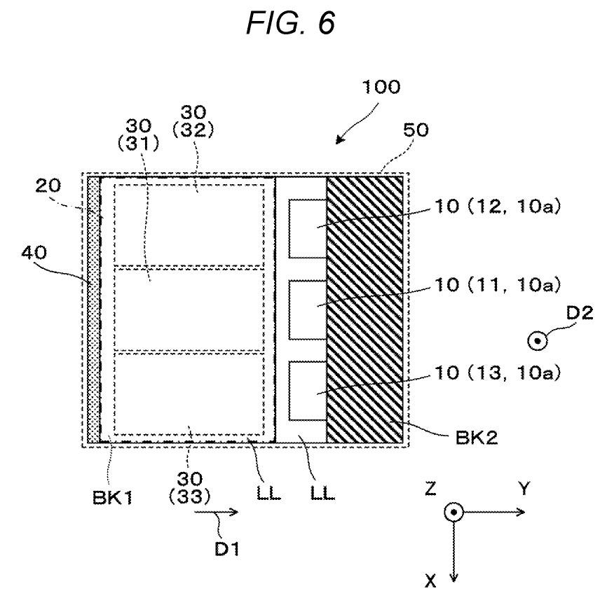

In the living body detection apparatus 100 according to the present embodiment, the light receiver 30 includes multiple light receivers 31 to 33, as shown in FIGS. 5 and 6. It is preferable that the number of the light receivers that constitute the light receiver 30 is equal to the number of the light emitters that constitute the light emitter 10.

Specifically, the light receiver 30 includes the first light receiver 31, which receives the light having the first wavelength band, the second light receiver 32, which receives the light having the second wavelength band, and the third light receiver 33, which receives the light having the third wavelength band. It is therefore unnecessary to drive the light emitter 10, for example, in a time-division manner, so that the power consumption can be suppressed. In addition, light reception accuracy of the light receiver 30 can be improved.

It is preferable that the first to third light emitters 11 to 13 and the first to third light receivers 31 to 33 are arranged so as to have the shortest optical paths corresponding to the respective wavelengths. Specifically, in the cross-sectional view taken along the line B-B and shown in FIG. 6, the light emitters that constitute the light emitter 10 are arranged in the following order in the X direction: the second light emitter 12; the first light emitter 11; and the third light emitter 13. The light receivers that constitute the light receiver 30 are arranged in the following order in the X direction: the second light receiver 32; the first light receiver 31; and the third light receiver 33. In the side cross-sectional view shown in FIG. 5, the plane parallel to the side cross section intersects with the first light emitter 11 and the first light receiver 31, intersects with the second light emitter 12 and the second light receiver 32, and intersects with the third light emitter 13 and the third light receiver 33.

### Third embodiment

A living body detection apparatus according to a third embodiment of the present disclosure will be described below. Note that the living body detection apparatus according to the third embodiment is a partly changed version of the living body detection apparatus according to the first embodiment, and portions common to those of the living body detection apparatus according to the first embodiment will not be described.

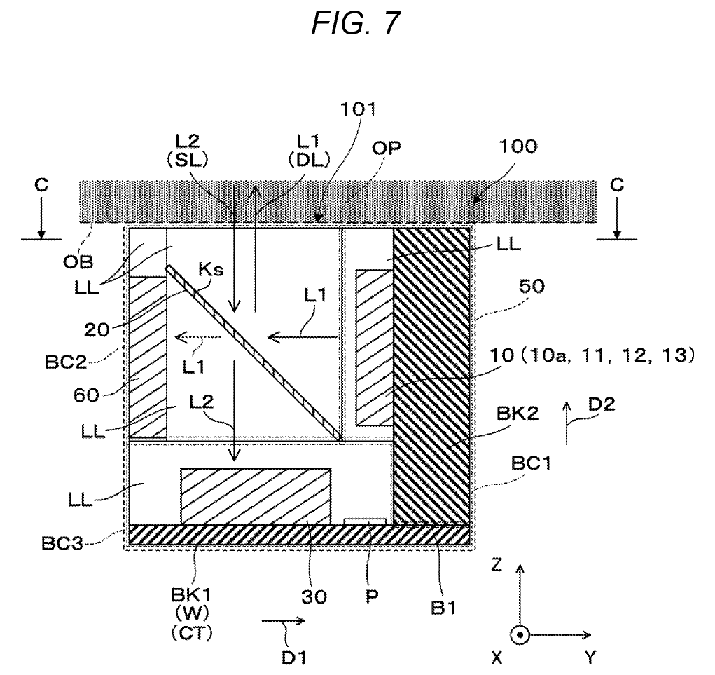

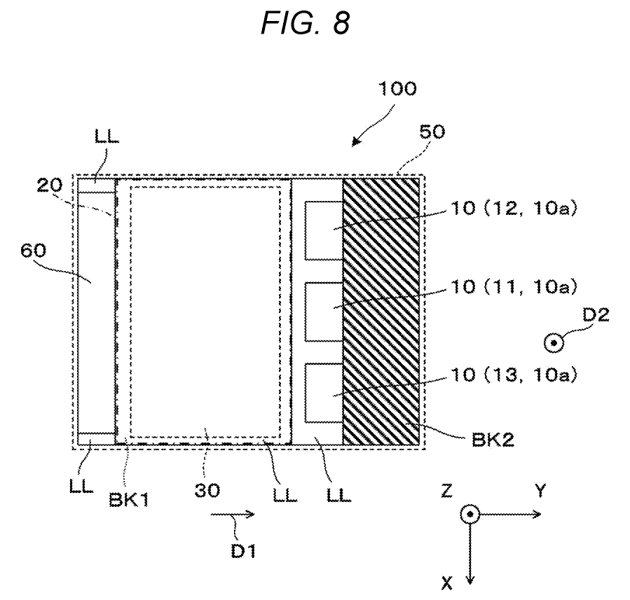

The living body detection apparatus 100 according to the present embodiment includes a reference light receiver 60, which receives the first light L1 having passed through the transmissive reflector 20, in place of the light absorbing layer 40 shown in FIG. 1, as shown in FIGS. 7 and 8. Using information on the first light L1 detected by the reference light receiver 60 to correct information on the second light L2 detected by the light receiver 30 allows suppression of noise, so that the accuracy of the detected living body information can be improved.

### Fourth embodiment

A living body detection apparatus according to a fourth embodiment of the present disclosure will be described below. Note that the living body detection apparatus according to the fourth embodiment is a partly changed version of the living body detection apparatus according to any of the second and third embodiments, and portions common to those of the living body detection apparatuses according to the second and third embodiments will not be described.

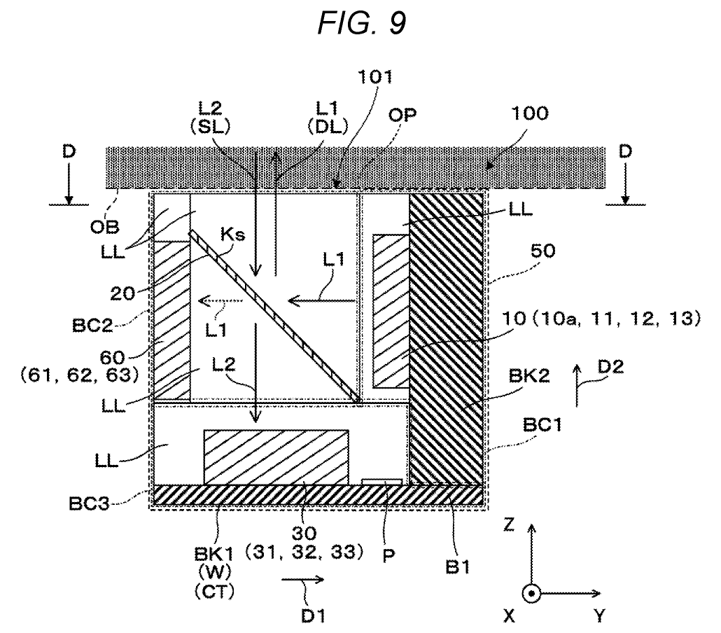

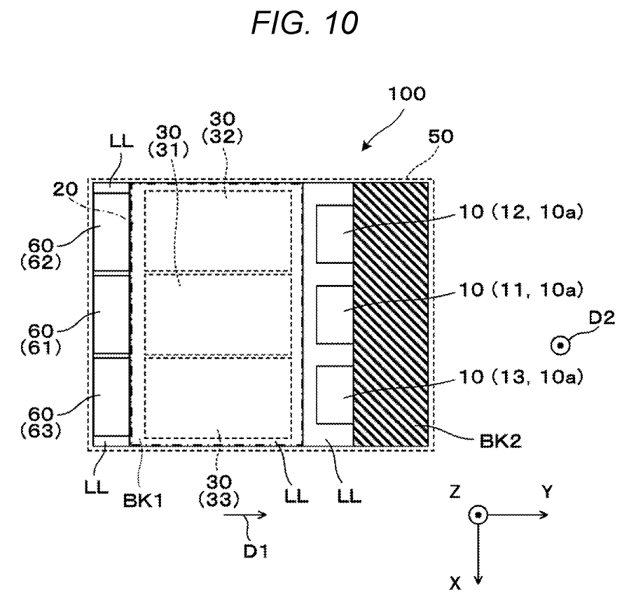

In the living body detection apparatus 100 according to the present embodiment, the reference light receiver 60 includes multiple reference light receivers, as shown in FIGS. 9 and 10. It is preferable that the number of the reference light receivers that constitute the reference light receiver 60 is equal to the number of the light emitters that constitute the light emitter 10 and the number of the light receivers that constitute the light receiver 30.

Specifically, the reference light receiver 60 includes a first reference light receiver 61, which receives the light having the first wavelength band, a second reference light receiver 62, which receives the light having the second wavelength band, and a third reference light receiver 63, which receives the light having the third wavelength band. It is unnecessary to drive the light emitter 10, for example, in a time-division manner, so that the power consumption can be suppressed. In addition, light reception accuracy of the reference light receiver 60 can be improved.

It is preferable that the first to third light emitters 11 to 13, the first to third light receivers 31 to 33, and the first to third reference light receivers 61 to 63 are arranged so as to have the shortest optical paths corresponding to the respective wavelengths.

### Fifth embodiment

A living body detection apparatus according to a fifth embodiment of the present disclosure will be described below. Note that the living body detection apparatus according to the fifth embodiment is a partly changed version of the living body detection apparatus according to the first embodiment, and portions common to those of the living body detection apparatus according to the first embodiment will not be described.

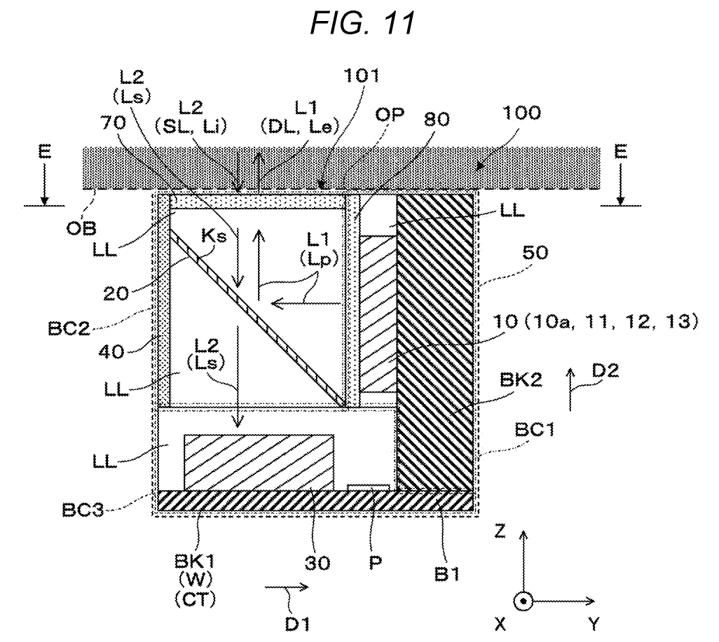

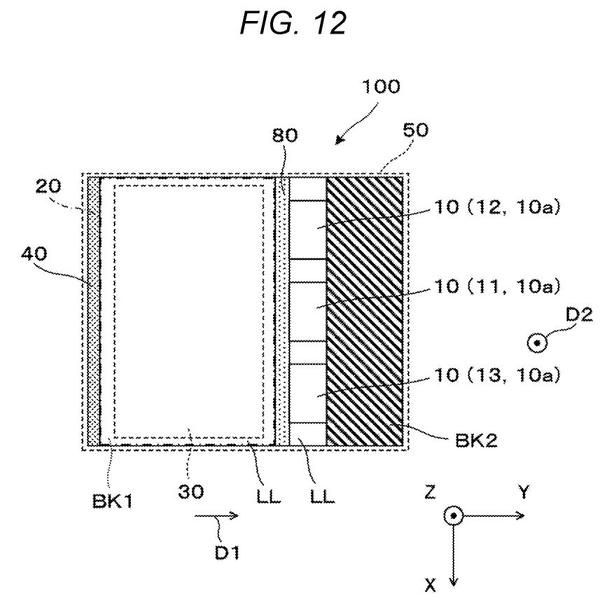

The living body detection apparatus 100 according to the present embodiment includes the first substrate BK1, the second substrate BK2, the light emitter 10, the transmissive reflector 20, the light receiver 30, the light absorbing layer 40, a quarter-wave plate 70, and a polarizing member 80, as shown in FIGS. 11 and 12.

The transmissive reflector 20 is, for example, a reflective polarizer plate or a polarizing beam splitter characterized by reflecting P-polarized light. The transmissive reflector 20 transmits a first polarized component (specifically, S-polarized light Ls) and reflects a second polarized component (specifically, P-polarized light Lp). The transmissive reflector 20 is configured, for example, with a dielectric multilayer film. The transmissive reflector 20 efficiently reflects the P-polarized light Lp when the first light L1 or the second light L2 contains the P-polarized light Lp, and efficiently transmits the S-polarized light Ls when the first light L1 or the second light L2 contains the S-polarized light Ls. The transmissive reflector 20 may be any element that selectively reflects the first light L1 or the second light L2 in accordance with the polarization direction thereof, and may, for example, be a multilayer film, a wire grid polarizer such as a wire grid film, or a reflective polarizing element using film stretching.

Although not shown, the transmissive reflector 20 may be a reflective polarizer plate characterized by reflecting S-polarized light. In this case, the first polarized component is the P-polarized light Lp, and the second polarized component is the S-polarized light Ls. That is, the transmissive reflector 20 transmits the first polarized component (P-polarized light Lp) and reflects the second polarized component (S-polarized light Ls).

The polarizing member 80 is provided in the optical path between the light emitter 10 and the transmissive reflector 20. The polarizing member 80 transmits the first light L1 emitted from the light emitter 10 and extracts, from the first light L1, the second polarized component, which is a predetermined polarized component reflected from the transmissive reflector 20. The polarizing member 80 is, for example, a wire-grid-type polarizer plate in which a fine grid made of metal such as aluminum is formed at a planar plate made, for example, of glass.

The quarter-wave plate 70 is provided in the opening OP on the side facing the detection target living body OB. The quarter-wave plate 70 converts the P-polarized light Lp, which is the second polarized component reflected from the transmissive reflector 20, into left-handed circularly polarized light Le, and converts right-handed circularly polarized light Li reflected from the detection target living body OB into the S-polarized light Ls, which is the first polarized component. In the example shown in FIG. 11, the quarter-wave plate 70 is a film member or a planar plate member made, for example, of a crystal having an optic axis between the X direction and the Z direction.

The optical path and the like of the living body detection apparatus 100 will be described below. The light emitter 10 emits the first light L1, which is the irradiation light DL, toward the detection target living body OB via the opening 101 or the opening OP, as shown in FIG. 11. The first light L1 emitted from the light emitter 10 travels via the polarizing member 80, which extracts the second polarized component, specifically, the P-polarized light Lp, which is incident on the transmissive reflector 20. The first light L1 that is the P-polarized light Lp and incident on the transmissive reflector 20 is reflected from the transmissive reflector 20 and enters the quarter-wave plate 70. The P-polarized light Lp having entered the quarter-wave plate 70 is converted into the left-handed circularly polarized light Le by the quarter-wave plate 70. The first light L1 that is the circularly polarized light Le is incident on the detection target living body OB. The first light L1 having entered the detection target living body OB is reflected from the detection target living body OB and becomes the second light L2. In this process, the second light L2 becomes the right-handed circularly polarized light Li as a result of the reflection off the detection target living body OB, in particular, hemoglobin.

The second light L2 that is the right-handed circularly polarized light Li reflected from the detection target living body OB enters the quarter-wave plate 70. The circularly polarized light Li having entered the quarter-wave plate 70 is converted into the S-polarized light Ls by the quarter-wave plate 70. The second light L2 that is the S-polarized light Ls passes through the transmissive reflector 20 and is received by the light receiver 30. The light receiver 30 receives the second light L2, which is the return light SL having returned from the detection target living body OB via the opening 101 or the opening OP, and outputs a signal corresponding to the intensity of the second light L2.

The living body detection apparatus 100 according to the present embodiment, in which the quarter-wave plate 70 uses specific polarized light as the first light L1 and the transmissive reflector 20 separates the two types of polarized light from each other, can prevent a decrease in the amount of light and further suppress the power consumption. The polarizing member 80 can extract the specific polarized light from the first light L1.

When the first light L1 emitted from the light emitter 10, travels via the polarizing member 80, and is incident on the transmissive reflector 20 is only the specific polarized light, for example, the P-polarized light Lp, the light absorbing layer 40 may be omitted.

The polarizing member 80 may be omitted. Even when the first light L1 emitted from the light emitter 10 is unpolarized light, the S-polarized light Ls of the first light L1 passing through the transmissive reflector 20 is absorbed by the light absorbing layer 40, so that noise can be suppressed.

The light emitter 10 may emit the specific polarized light. In this case, a half-wave plate may be provided in place of the polarizing member 80 to convert the first light L1 into light having the predetermined polarization direction.

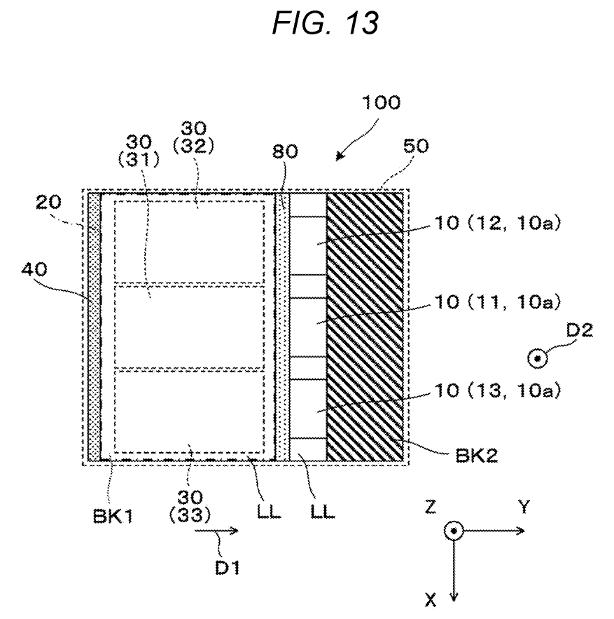

In the living body detection apparatus 100, the light receiver 30 may include the multiple light receivers 31 to 33, as shown in FIG. 13.

### Sixth embodiment

A living body detection apparatus according to a sixth embodiment of the present disclosure will be described below. Note that the living body detection apparatus according to the sixth embodiment is a partly changed version of the living body detection apparatus according to the first embodiment, and portions common to those of the living body detection apparatus according to the first embodiment will not be described.

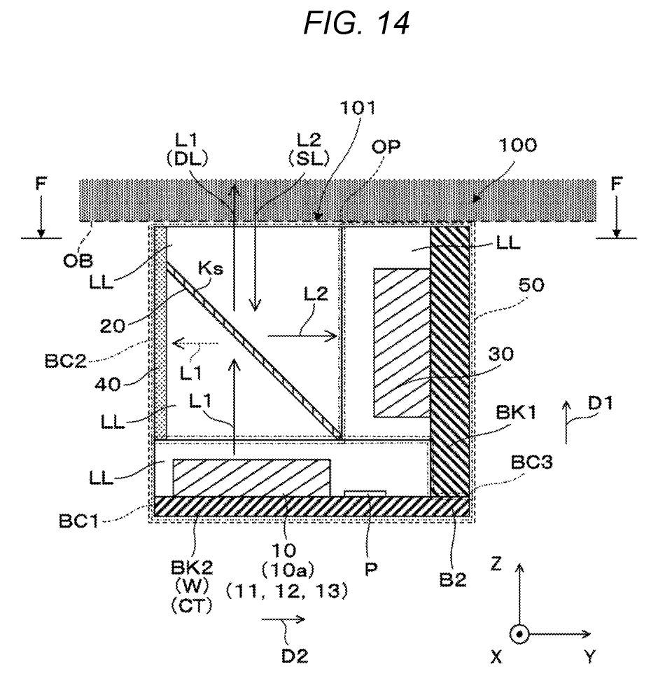

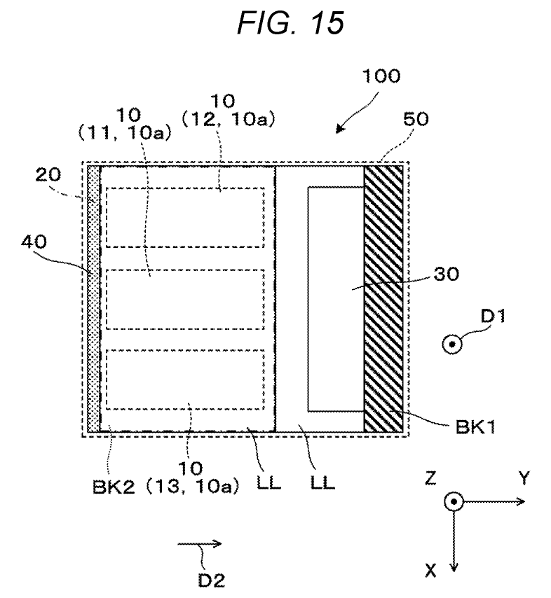

In the living body detection apparatus 100 according to the present embodiment, the arrangement of the light emitter 10 and the light receiver 30 is reversed as compared with the living body detection apparatus 100 shown in FIG. 1, as shown in FIGS. 14 and 15.

The living body detection apparatus 100 includes the first substrate BK1, the second substrate BK2, the light emitter 10, the transmissive reflector 20, the light receiver 30, and the light absorbing layer 40.

In the present embodiment, the first direction D1 is the Z direction and corresponds to the upward-downward direction in the plane of view of FIG. 14. The second direction D2 is the Y direction and corresponds to the rightward-leftward direction in the plane of view of FIG. 14. In the example shown in FIG. 14, the first substrate BK1 extends perpendicularly from an end portion B2 on one side of the second substrate BK2 in the second direction D2.

The light emitter 10 is provided at the second substrate BK2. The light receiver 30 is provided at the first substrate BK1. The transmissive reflector 20 is obliquely provided at a position where the first light L1 incident from the light emitter 10 and the second light L2 to be incident on the light receiver 30 intersect with each other.

The living body detection apparatus 100 is provided with the light transmissive member LL in the internal space excluding the first substrate BK1, the second substrate BK2, the light emitter 10, the transmissive reflector 20, the light receiver 30, and the light absorbing layer 40. The transmissive reflector 20 in the inclining posture is surrounded by the light transmissive member LL. The light emitter 10 and the light receiver 30 are embedded and disposed in outer portions of the light transmissive member LL. The first substrate BK1, the second substrate BK2, and the light absorbing layer 40 are disposed so as to cover the outer side of the light transmissive member LL.

In the present embodiment, the transmissive reflector 20 receives the first light L1 from the light emitter 10, transmits part of the first light L1 toward the detection target living body OB, receives the second light L2 from the detection target living body OB, and reflects part of the second light L2. The second light L2 reflected from the transmissive reflector 20 is incident on the light receiver 30.

In the present embodiment, the light emitter 10 is disposed at a position shifted from the transmissive reflector 20 in the first direction D1, specifically, in the −Z direction or on the lower side of the plane of view of FIG. 14. The light receiver 30 is disposed at a position shifted from the transmissive reflector 20 in the second direction D2, specifically, in the +Y direction or on the right side of the plane of view of FIG. 14.

The light absorbing layer 40 is disposed on the side opposite the light receiver 30 with the transmissive reflector 20 interposed therebetween. The light absorbing layer 40 absorbs the first light L1 reflected from the transmissive reflector 20. The light absorbing layer 40 prevents the first light L1 emitted from the light emitter 10 and reflected from the transmissive reflector 20 from being directly incident on the transmissive reflector 20 again. Noise can thus be suppressed. Note that, in the embodiment, the light absorbing layer 40 may be omitted.

The circuit substrate W is mounted, for example, on the second substrate BK2. The circuit substrate W may be mounted on the first substrate BK1, may be mounted on both the first substrate BK1 and the second substrate BK2, or may be separate from the first substrate BK1 and the second substrate BK2.

The optical path and the like of the living body detection apparatus 100 will be described below. The light emitter 10 emits the first light L1, which is the irradiation light DL, toward the detection target living body OB via the opening 101 or the opening OP, as shown in FIG. 14. The first light L1 emitted from the light emitter 10 passes through the transmissive reflector 20 by about 50% and is incident on the detection target living body OB. The remaining 50% of the first light L1 is reflected from the transmissive reflector 20 and absorbed by the light absorbing layer 40. The first light L1 having entered the detection target living body OB is reflected from the detection target living body OB and becomes the second light L2.

The second light L2 reflected from the detection target living body OB is reflected from the transmissive reflector 20 by about 50% and is received by the light receiver 30. The light receiver 30 receives the second light L2, which is the return light SL having returned from the detection target living body OB via the opening 101 or the opening OP, and outputs a signal corresponding to the intensity of the second light L2.

According to the living body detection apparatus 100 described above, since the light emitter 10 is so disposed that the light therefrom passes through the transmissive reflector 20, and the light receiver 30 is disposed so as to receive the light reflected from the transmissive reflector 20, one port (specifically, opening 101) serves as the light entrance and the light exit, so that the area of the surface where the living body detection apparatus 100 comes into contact with the detection target living body OB can be reduced. The size of the living body detection apparatus can thus be reduced.

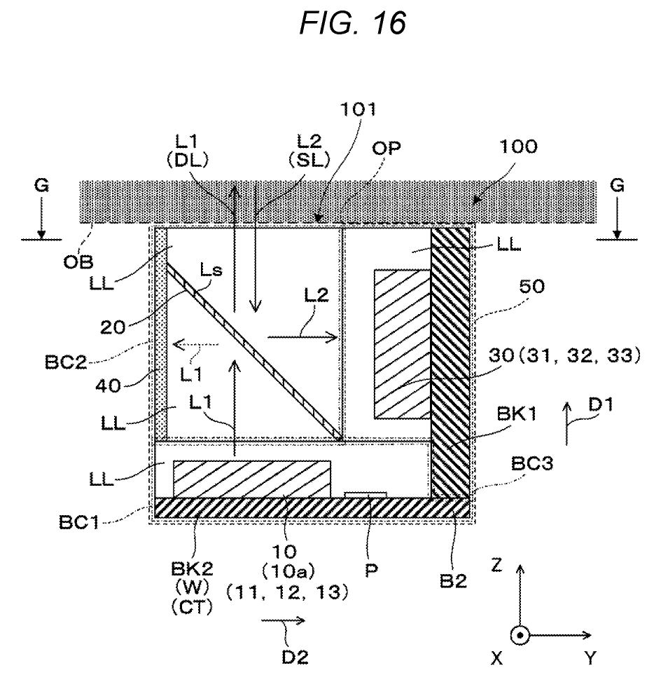

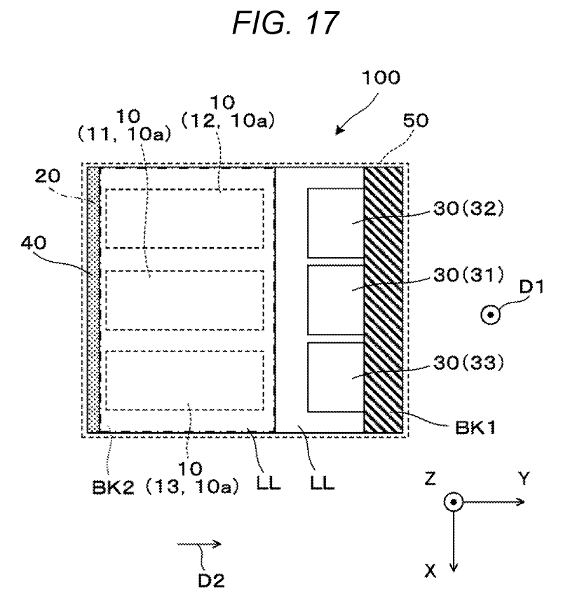

Note in the living body detection apparatus 100 that the light receiver 30 may include the multiple light receivers 31 to 33, as shown in FIGS. 16 and 17.

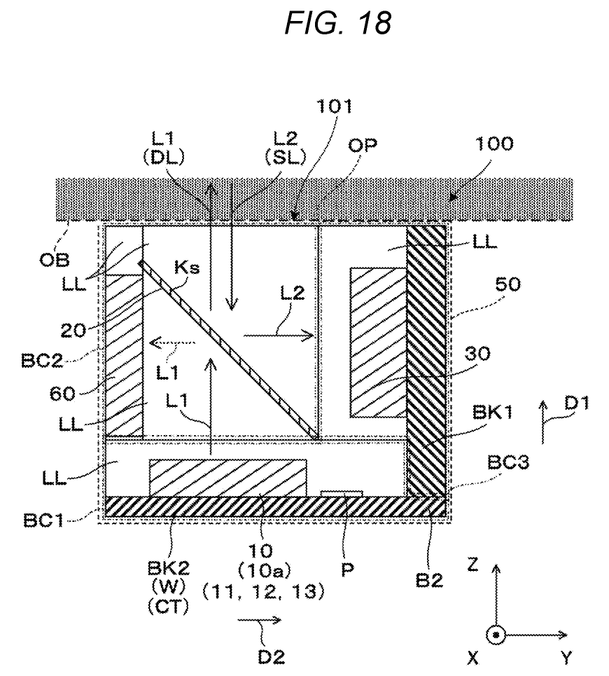

The living body detection apparatus 100 may include one or more reference light receivers that constitute the reference light receiver 60 and receive the first light L1 reflected from the transmissive reflector 20, as shown in FIG. 18.

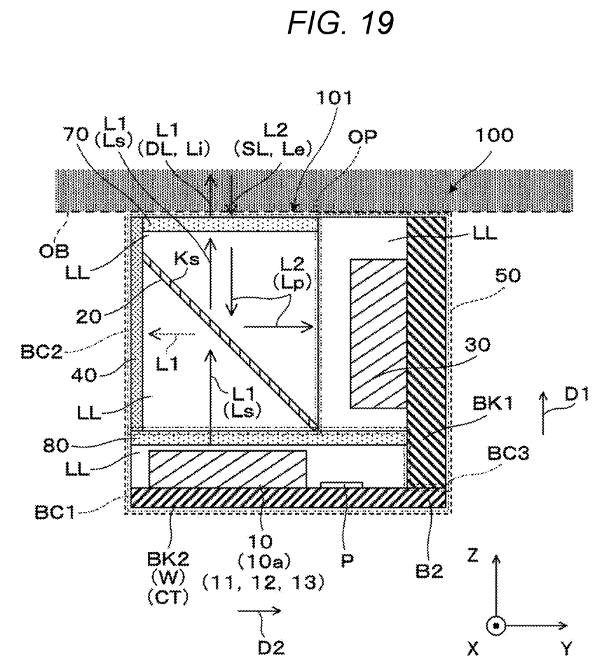

In the living body detection apparatus 100, the transmissive reflector 20 may be a reflective polarizer plate, and may include the quarter-wave plate 70 and the polarizing member 80, as shown in FIG. 19. In the example shown in FIG. 19, the transmissive reflector 20 is, for example, a reflective polarizer plate characterized by reflecting P-polarized light. The transmissive reflector 20 transmits the first polarized component (specifically, S-polarized light Ls) and reflects the second polarized component (specifically, P-polarized light Lp).

### Other Items

The structures described above are presented by way of example, and can be changed in various manners to the extent that the same functions can be achieved.

For example, the sizes, shapes, numbers, arrangements, and other factors of the light emitter 10, the light receiver 30, the reference light receiver 60, and the like can be changed as appropriate.

The polarization characteristics of the transmissive reflector 20, the first polarized component, and the second polarized component are presented by way of example and can be changed as appropriate.

The interior of the living body detection apparatus 100 may be an air layer in place of the light transmissive member LL. In this case, a side surface of the transmissive reflector 20 is fixed, for example, to the housing member 50.

### Summary of present disclosure

The present disclosure will be summarized below as additional remarks.

### Additional Remark 1

A living body detection apparatus including: a light emitter configured to emit first light; a light receiver configured to receive second light that is the first light reflected from a detection target living body; and a transmissive reflector on which the first light is incident, which is configured to reflect part of the first light toward the detection target living body, on which the second light is incident, and which is configured to transmit part of the second light, wherein the second light passing through the transmissive reflector is incident on the light receiver.

Since the light emitter is disposed in the reflection direction of the transmissive reflector and the light receiver is disposed in the transmission direction, one port serves as the light entrance and the light exit, so that the area of the surface where the living body detection apparatus comes into contact with the detection target living body can be reduced. The size of the living body detection apparatus can thus be reduced.

### Additional Remark 2

A living body detection apparatus including: a light emitter configured to emit first light; a light receiver configured to receive second light that is the first light reflected from a detection target living body; and a transmissive reflector on which the first light is incident, which is configured to transmit part of the first light toward the detection target living body, on which the second light is incident, and which is configured to reflect part of the second light, wherein the second light reflected from the transmissive reflector is incident on the light receiver. Since the light emitter is disposed in the reflection direction of the transmissive reflector and the light receiver is disposed in the transmission direction, one port serves as the light entrance and the light exit, so that the area of the surface where the living body detection apparatus comes into contact with the detection target living body can be reduced. The size of the living body detection apparatus can thus be reduced.

### Additional Remark 3

The living body detection apparatus according to Additional Remark 1 or 2, wherein the light emitter includes a first light emitter configured to emit light having a first wavelength band, a second light emitter configured to emit light having a second wavelength band, and a third light emitter configured to emit light having a third wavelength band. Multiple pieces of living body information can be detected by controlling the operation of driving the first to third light emitters.

### Additional Remark 4

The living body detection apparatus according to Additional Remark 3, wherein the light receiver includes a first light receiver configured to receive the light having the first wavelength band, a second light receiver configured to receive the light having the second wavelength band, and a third light receiver configured to receive the light having the third wavelength band.

It is unnecessary to drive the light emitter, for example, in a time-division manner, so that the power consumption can be suppressed. In addition, light reception accuracy of the light receiver can be improved.

### Additional Remark 5

The living body detection apparatus according to any one of Additional Remarks 1 to 4, further including: a first substrate extending in a first direction; and a second substrate extending in a second direction that intersects with the first direction, wherein the light receiver is provided at the first substrate, and the light emitter is provided at the second substrate.

The configuration in which the first substrate and the second substrate intersect with each other allows efficient arrangement of the light emitter and the light receiver.

### Additional Remark 6

The living body detection apparatus according to any one of Additional Remarks 1 to 5, further including a light absorbing layer configured to absorb the first light passing through the transmissive reflector.

The light absorbing layer absorbs the first light having passed through the transmissive reflector, so that noise can be suppressed.

### Additional Remark 7

The living body detection apparatus according to any one of Additional Remarks 1 to 5, further including a light absorbing layer configured to absorb the first light reflected from the transmissive reflector.

The light absorbing layer absorbs the first light reflected from the transmissive reflector, so that noise can be suppressed.

### Additional Remark 8

The living body detection apparatus according to any one of Additional Remarks 1 to 5, further including a reference light receiver configured to receive the first light passing through the transmissive reflector.

Using information on the first light detected by the reference light receiver to correct information on the second light detected by the light receiver allows suppression of noise, so that the accuracy of the detected living body information can be improved.

### Additional Remark 9

The living body detection apparatus according to any one of Additional Remarks 1 to 5, further including a reference light receiver configured to receive the first light reflected from the transmissive reflector.

Using information on the first light detected by the reference light receiver to correct information on the second light detected by the light receiver allows suppression of noise, so that the accuracy of the detected living body information can be improved.

### Additional Remark 10

The living body detection apparatus according to any one of Additional Remarks 8 and 9, wherein the reference light receiver includes a first reference light receiver configured to receive the light having the first wavelength band, a second reference light receiver configured to receive the light having the second wavelength band, and a third reference light receiver configured to receive the light having the third wavelength band.

It is unnecessary to drive the light emitter, for example, in a time-division manner, so that the power consumption can be suppressed. In addition, light reception accuracy of the reference light receiver can be improved.

### Additional Remark 11

The living body detection apparatus according to any one of Additional Remarks 1 to 7, further including a quarter-wave plate provided at an opening on a side facing the detection target living body, wherein the transmissive reflector is configured to transmit a first polarized component and reflect a second polarized component.

The configuration described above, in which the quarter-wave plate uses specific polarized light as the first light and the transmissive reflector separates the two types of polarized light from each other, can prevent a decrease in the amount of light and further suppress the power consumption.

### Additional Remark 12

The living body detection apparatus according to Additional Remark 11, further including a polarizing member provided in an optical path between the light emitter and the transmissive reflector.

The polarizing member can extract the specific polarized light from the first light.

### Additional Remark 13

The living body detection apparatus according to any one of Additional Remarks 1 to 12, wherein the light emitter includes a surface-emission-type light emitting element.

Therefore, even when the light emitter performs multi-color emission, it is unnecessary to stack light emitting elements to form a multi-wavelength light emitter, so that the size of the light emitter can be reduced.

What is claimed is:

1. A living body detection apparatus comprising:
a light emitter configured to emit first light;
a light receiver configured to receive second light that is the first light reflected from a detection target living body; and
a transmissive reflector on which the first light is incident, which is configured to reflect part of the first light toward the detection target living body, on which the second light is incident, and which is configured to transmit part of the second light,
wherein the second light passing through the transmissive reflector is incident on the light receiver.

2. A living body detection apparatus comprising:
a light emitter configured to emit first light;
a light receiver configured to receive second light that is the first light reflected from a detection target living body; and
a transmissive reflector on which the first light is incident, which is configured to transmit part of the first light toward the detection target living body, on which the second light is incident, and which is configured to reflect part of the second light,
wherein the second light reflected from the transmissive reflector is incident on the light receiver.

3. The living body detection apparatus according to claim 1, wherein
the light emitter includes a first light emitter configured to emit light having a first wavelength band, a second light emitter configured to emit light having a second wavelength band, and a third light emitter configured to emit light having a third wavelength band.

4. The living body detection apparatus according to claim 3, wherein
the light receiver includes a first light receiver configured to receive the light having the first wavelength band, a second light receiver configured to receive the light having the second wavelength band, and a third light receiver configured to receive the light having the third wavelength band.

5. The living body detection apparatus according to claim 1, further comprising:
a first substrate extending in a first direction; and a second substrate extending in a second direction that intersects with the first direction,
wherein the light receiver is provided at the first substrate, and the light emitter is provided at the second substrate.

6. The living body detection apparatus according to claim 1, further comprising
a light absorbing layer configured to absorb the first light passing through the transmissive reflector.

7. The living body detection apparatus according to claim 2, further comprising
a light absorbing layer configured to absorb the first light reflected from the transmissive reflector.

8. The living body detection apparatus according to claim 1, further comprising
a reference light receiver configured to receive the first light passing through the transmissive reflector.

9. The living body detection apparatus according to claim 2, further comprising
a reference light receiver configured to receive the first light reflected from the transmissive reflector.

10. The living body detection apparatus according to claim 8, wherein
the reference light receiver includes a first reference light receiver configured to receive the light having the first wavelength band, a second reference light receiver configured to receive the light having the second wavelength band, and a third reference light receiver configured to receive the light having the third wavelength band.

11. The living body detection apparatus according to claim 1, further comprising
a quarter-wave plate provided at an opening on a side facing the detection target living body,
wherein the transmissive reflector is configured to transmit a first polarized component and reflect a second polarized component.

12. The living body detection apparatus according to claim 11, further comprising
a polarizing member provided in an optical path between the light emitter and the transmissive reflector.

13. The living body detection apparatus according to claim 1, wherein
the light emitter includes a surface-emission-type light emitting element.
